(19) United States
(12) Patent Application Publication
(10) Pub. No.: US 2024/0277467 A1
(43) Pub. Date: Aug. 22, 2024

(54) METHODS AND IMPLANTABLE PROSTHESIS FOR RECONSTRUCTION AND/OR AUGMENTATION OF AN ANATOMICAL FEATURE

(71) Applicant: Davol Inc., Warwick, RI (US)

(72) Inventors: Ian K. Parker, Bristol, RI (US); Jeremy Griffin, North Attleboro, MA (US); Evan Gray, Richmond, RI (US); Angel Pagan-Ortiz, Providence, RI (US)

(73) Assignee: Davol Inc., Warwick, RI (US)

(21) Appl. No.: 18/571,704

(22) PCT Filed: Jun. 30, 2022
(86) PCT No.: PCT/US2022/035716

§ 371 Date: Dec. 18, 2023

Related U.S. Application Data
(60) Provisional application No. 63/218,231, filed on Jul. 2, 2021.
(60) Provisional application No. 63/218,236, filed on Jul. 2, 2021.

(51) Int. Cl. A61F 2/12; A61F 2/00

(52) U.S. Cl. CPC A61F 2/12; A61F 2/0077; A61F 2002/0086; A61F 2230/0067; A61F 2240/001

(57) ABSTRACT

An implantable prosthesis having a tissue infiltratable body of biocompatible material includes a plurality of body components arranged radially or circumferentially about a longitudinal axis of the body extending from a proximal end to a distal end of the body. The longitudinal axis may define a central axis of the prosthesis. Each body component includes an outer layer defining a cavity. The outer layer may be substantially conically shaped and/or pleated. A filler may be disposed within the cavity. The body may be attached to abase. The body is configured to augment and/or reconstruct an anatomical shape of a human breast.

### RELATED APPLICATIONS

Foreign priority benefits are claimed under 35 U.S.C. § 119(a)-(d) or 35 U.S.C. § 365(b) of U.S. application Nos. 63/218,231, filed Jul. 2, 2021, and 63/218,236, filed Jul. 2, 2021, each of which is incorporated herein by reference.

### FIELD

The present disclosure relates to an implantable prosthesis, and more particularly to a prosthesis for reconstruction and/or augmentation of an anatomical feature, including a human breast.

### BACKGROUND

Breast reconstruction is primarily performed following breast cancer diagnosis and surgical treatment. However, a growing number of patients are choosing breast reconstruction as a prophylactic option in response to genetic testing results which may indicate an individual being at high risk for breast cancer.

Breast reconstruction can be generally categorized as autologous and non-autologous. For autologous reconstruction, a patient's own tissue is harvested from another part of their body and then used to reconstruct the breast. For non-autologous reconstruction, an artificial implant, such as a saline, silicone, or gel implant, is employed to reconstruct the breast mound.

Autologous reconstruction generally involves harvesting a tissue flap from the abdominal region of a patient. This procedure can maintain vascular supply to the patient's tissue, and generally provides an aesthetically pleasing outcome for the patient. However, such a procedure can be time consuming, with possible microsurgery to reconnect the vascular supply, and require a relatively longer recovery time. It can also create functional deficits and weakness in the area from which the tissue has been removed. This technique may not be available to some patients who lack belly volume or cannot afford a reduction of muscle mass.

Non-autologous reconstruction, which involves a wide majority of breast reconstructive procedures, may employ single stage or intermediate reconstruction procedures, or dual stage reconstruction procedures. A mastectomy and reconstruction of the breast can be performed at the same time (single stage) or staged over multiple procedures (dual stage). In each procedure, a breast implant is typically placed below the pectoral muscle, i.e., sub-pectoral, to mask the implant from being seen through the skin and cover the relatively stiff implant with muscle.

In a single stage procedure, breast tissue is completely dissected and removed after a small incision is made under the breast. The pectoral muscle is subsequently detached at its lower end and the sub-muscular plane is developed to create a sub-pectoral pocket with sufficient size to accommodate the implant. An acellular dermal matrix (ADM) is typically employed to reattach the muscle and add reinforcement under the implant.

Single stage procedures generally do not provide much control over the cosmetic outcome because it cannot be adjusted over time. These procedures could also potentially result in tissue necrosis should the implant be too large for the size of the sub-muscular pocket.

In a dual stage procedure, the initial surgical stage is similar to the single stage procedure. However, rather than placing an implant into the sub-muscular pocket, an ADM is initially placed in the pocket and followed by placement of a tissue expander. The ADM is manipulated as needed to accommodate the tissue expander and then fixated into place. Following the initial surgical stage, the tissue expander is filled over multiple post-surgical office visits to slowly expand the space below the pectoral muscle to create a pocket. Once a sufficiently sized pocket is formed, typically six months after the initial procedure, a second surgical procedure is performed to remove the expander and insert the breast implant in the sub-muscular pocket created by the expander.

A more recent trend in breast reconstruction involves pre-pectoral placement of an implant on top of the pectoral muscle to avoid creation of a sub-muscular pocket. During such procedures, the implant is typically wrapped completely with ADM rather than using the ADM as a sling which only partially covers the implant.

It has been reported that the breast is shaped by a three-dimensional, fibrofatty fascial system. Two layers of this system surround the corpus mammae and fuse together around and anchor it to the chest wall in a structure identified as the circum-mammary ligament (CML). The CML, which defines the perimeter of the breast, is a 3D, roughly circular structure composed of superficial fascia collagen fibers that encase a ring of fat and attach it to the deep fascia of the chest, as a circular zone of adherence.

It is an object of the disclosure to provide methods and a prosthesis for augmenting and/or reconstructing a breast.

### SUMMARY

The present disclosure relates to methods and an implantable prosthesis for augmenting and/or reconstructing an anatomical feature, including a human breast.

According to one embodiment, an implantable prosthesis includes a tissue infiltratable body of biocompatible material, the body including a proximal end and a distal end spaced from the proximal end. The body being configured to augment and/or reconstruct an anatomical shape of a human breast. The body also includes a plurality of body components circumferentially arranged about a longitudinal axis of the body extending from the proximal end to the distal end. Each of the plurality of body components including an outer layer defining a cavity, the outer layer having a conical shape.

According to another embodiment, an implantable prosthesis includes a tissue infiltratable body of biocompatible material, the body having a proximal end and a distal end spaced form the proximal end. The body being configured to augment and/or reconstruct an anatomical shape of a human breast. The body also includes a plurality of body components arranged about a longitudinal axis of the body extending from the proximal end to the distal end, wherein each of the body components includes an outer layer defining a cavity, the outer layer being pleated, and a base attached to the body.

According to another embodiment, a method of fabricating an implantable prosthesis is disclosed. The method includes the act of (a) providing a plurality of body components, each of the plurality of body components including an outer layer defining a cavity. The outer layer has a conical shape and/or the outer layer is pleated. The method also includes the acts of (b) arranging the plurality of body components circumferentially about a longitudinal axis of the prosthesis and in an outward radial direction to form a body configured to augment and/or reconstruct an anatomical shape of a human breast, and (c) securing the plurality of body components together.

According to another embodiment, an implantable prosthesis includes a tissue infiltratable body of biocompatible material, the body including a proximal end and a distal end spaced from the proximal end. The body also includes a plurality of body components arranged about a longitudinal axis of the body extending from the proximal end to the distal end, wherein each of the body components includes an outer layer defining a cavity and a filler located within the cavity, and a base attached to the prosthesis body.

According to another embodiment, a method of fabricating an implantable prosthesis is disclosed. The method includes an act of (a) providing a plurality of body components, each of the plurality of body components including an outer layer defining a cavity. The outer layer includes a filler material within the cavity. The method also includes acts of (b) arranging the plurality of body components circumferentially about a longitudinal axis of the prosthesis and in an outward radial direction, and (c) securing the plurality of body components together.

It should be appreciated that the foregoing concepts, and additional concepts discussed below, may be arranged in any suitable combination, as the present disclosure is not limited in this respect.

The foregoing and other aspects, embodiments, and features of the present teachings can be more fully understood from the following description in conjunction with the accompanying drawings.

### BRIEF DESCRIPTION OF DRAWINGS

Aspects of the disclosure are described below, by way of example, with reference to the accompanying drawings, and wherein:

FIGS. 1A-1F are schematic illustrations of a breast reconstruction procedure;

FIG. 2 is a perspective view of an implantable prosthesis for breast reconstruction and/or augmentation according to an embodiment of the present disclosure;

FIG. 3 is a top view of the prosthesis of FIG. 2;

FIG. 4 is a bottom perspective view of a body of the prosthesis of FIG. 2;

FIG. 5 is a bottom view of a base of the prosthesis of FIG. 2;

FIGS. 6 and 7 are top and bottom perspective views of a body component of an implantable prosthesis according to one embodiment;

FIG. 8 is a top perspective view of a body component of an implantable prosthesis according to another embodiment;

FIG. 9A is an exploded view of a body component of an implantable prosthesis according to one embodiment and FIG. 9B is the assembled body component of FIG. 9A;

FIGS. 10 and 11 are top perspective views of a body component according to another embodiment;

FIG. 12 is a top perspective view of a body component of an implantable prosthesis according to another embodiment;

FIG. 13 shows the body component of FIG. 12 in an unassembled configuration;

FIG. 14 shows a body component of an implantable prosthesis according to another embodiment;

FIG. 15A is an exploded view of a body component of an implantable prosthesis according to another embodiment and FIG. 15B is the assembled body component of FIG. 15A;

FIG. 16A is an exploded view of a body component of an implantable prosthesis according to another embodiment and FIG. 16B is the assembled body component of FIG. 16A;

FIG. 17A is an exploded view of a body component of an implantable prosthesis according to another embodiment and FIG. 17B is the assembled body component of FIG. 17A;

FIG. 18A is an exploded view of a body component of an implantable prosthesis according to another embodiment and FIG. 18B is the assembled body component of FIG. 18A;

FIG. 19 is a schematic illustration of an implantable prosthesis according one embodiment;

FIG. 20 is a schematic illustration of an implantable prosthesis according to another embodiment;

FIG. 21 is a schematic illustration of an implantable prosthesis according to another embodiment;

FIG. 22 is a schematic illustration of an implantable prosthesis according to another embodiment; and

FIG. 23 is a top view of an implantable prosthesis according to another embodiment.

### DESCRIPTION

The present disclosure is directed to an implantable prosthesis particularly suited for augmenting and/or reconstructing an anatomical feature, including a human breast. However, the prosthesis may be suitable for mending anatomical defects in, and weaknesses of, soft tissue and muscle walls or other anatomical regions. For purposes herein, the phrase “mending a defect” includes acts of repairing, augmenting, and/or reconstructing a defect and/or a potential defect.

For ease of understanding, and without limiting the scope of the disclosure, the prosthesis is described below particularly in connection with breast reconstruction. It should be understood, however, that the prosthesis is not so limited and may be employed in other anatomical procedures, as should be apparent to one of skill in the art. For example, and without limitation, the prosthesis, or aspects of the prosthesis, may be employed for hernias, chest or abdominal wall reconstruction, or large defects, such as those that may occur in obese patients. The prosthesis may include one or more features, each independently or in combination, contributing to such attributes.

As will be described further below, the prosthesis may have particular application with pre-pectoral breast reconstruction procedures.

### Pre-Pectoral Breast Reconstruction

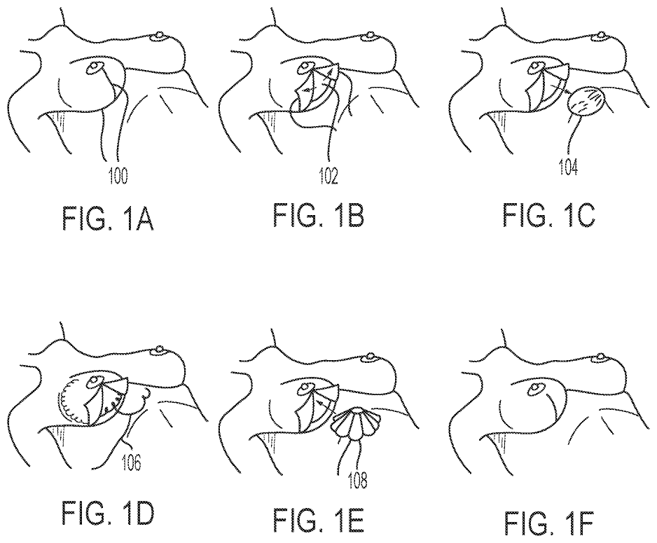

One embodiment of a pre-pectoral breast reconstruction procedure is illustrated in FIGS. 1A-1F.

As illustrated in FIG. 1A, an initial incision 100 may be made to form tissue flaps 102 on a lower portion of the breast. The flaps 102 may be spread apart to provide access to the breast tissue and permit removal of a tumor or other growth, as illustrated in FIG. 1B. It is desirable to preserve skin and the nipple areola complex, when possible.

As illustrated in FIG. 1C, the corpus mammae 104 may be removed, along with the bases of the Cooper's ligaments. Thereafter, as illustrated in FIG. 1D, the circum-mammary ligament (CML) 106 may be tightened to reestablish a desirable diameter base for the breast. For example, the CML may be tightened to correspond with the breast's pre-op diameter base, or tightened even more should the CML have been stretched and loosened over time due to aging and/or other factors.

As illustrated, the CML may be tightened using a purse string suturing technique in which a running suture is placed around and/or through the CML and then pulled upwardly to cinch-up the tissue and tighten the base. However, other procedures are contemplated for reestablishing the base of the breast.

Once the CML has been tightened, a prosthetic implant 108 may be inserted, as illustrated in FIG. 1E, into the anatomical cavity created by the removal of the corpus mammae. Prior to insertion, the implant may be coated with a fat graft which has been lipo-aspirated from the patient. In some embodiments, the fat graft may help soften the prosthesis and/or provide seeding for new fat and/or tissue being formed in and about the prosthesis. The fat graft may also reduce the potential for fluid to fill the space created by the removal of tissue during the procedure. However, it is to be appreciated that the implant does not need to be coated with a fat graft prior to insertion.

The fat graft, if desired, may be harvested from the patient using standard lipo-aspiration techniques. The aspirated fat may be processed on site to remove oils and provide a more purified fat for the procedure. The processed fat may be applied to various surfaces and within various voids of the prosthesis using a syringe or similar device, although other techniques are contemplated for coating the prosthesis.

Once inserted, the implant may be secured to the CML using sutures or other fasteners placed along a base of the device. Following implantation of the prosthesis, the incision may be closed without tension, as illustrated in FIG. 1F.

Following the initial reconstruction procedure, fat grafting into the breast may be performed with one or more procedures over time to achieve a desired shape and/or feel for the breast and/or symmetry between both breasts. For example, in some embodiments, process fat may be injected into the breast and into the various voids of the prosthesis.

### Prosthesis and Fabrication Concepts

The disclosure is more particularly directed to an implantable prosthesis, such as for breast reconstruction and/or augmentation. According to one aspect, the implantable prosthesis may include a tissue infiltratable body of a biocompatible for placement within an anatomical space.

In some embodiments, the body may have a three-dimensional configuration, such as to reconstruct and/or augment the anatomical shape of a breast. The prosthesis may be configured to encourage the ingrowth of fat and/or tissue to fill open spaces within and about the prosthesis. The prosthesis may employ a structure having a plurality of voids including, but not limited to, chambers, cavities, channels, openings, pockets, and/or pores, to allow fat and/or tissue to fill and pass through the structure and fill-out the reconstructed or augmented breast. The overall desired structure of the prosthesis may employ various constructs for its fabrication in an efficient manner.

In some embodiments, the prosthesis may include a body fabricated using a plurality of body components which can be assembled to create the desired shape of the prosthesis. In some embodiments, a body component may have a three-dimensional (3D) configuration and include one or more cavities for receiving fat and/or tissue. In some embodiments, the body component may include a filler material within a cavity to add bulk to the prosthesis and/or to create voids for receiving the fat and/or tissue. In some embodiments the body component may have a two-dimensional (2D) configuration that can be transformed into the 3D configuration during assembly. In this manner, the individual components may be fabricated in a relatively less complex manner and then assembled to create the desired arrangement (e.g., shape and/or number of voids) of the prosthesis. The body components may also be configured to provide the prosthesis with a desired amount of resilience and support.

In some embodiments, the prosthesis may include a proximal end, such as a base or bottom surface, for placement against fascia, such as the pectoral muscle. In some embodiments, in some embodiments, a base may be attached to the prosthesis body. In some embodiments, the base may be planar or have a generally rounded shape including, but not limited to, a convex shape. Such a configuration may facilitate positioning and placement of the prosthesis against the pectoral muscle and within the circum-mammary ligament, which may establish the position of the prosthesis on the chest wall. In this manner, the interaction between the implant and the anatomy may create a ball and socket-like arrangement.

In some embodiments, the plurality of body components may be arranged radially and/or circumferentially about a longitudinal axis of the prosthesis. In some embodiments, the longitudinal axis may extend from a proximal end to a distal end of the prosthesis. In some embodiments, the longitudinal axis may define a central axis of the prosthesis body. In some embodiments, the longitudinal axis may be spaced (e.g., offset) from the central axis of the body. In some embodiments, the longitudinal axis may be parallel to and spaced from the central axis.

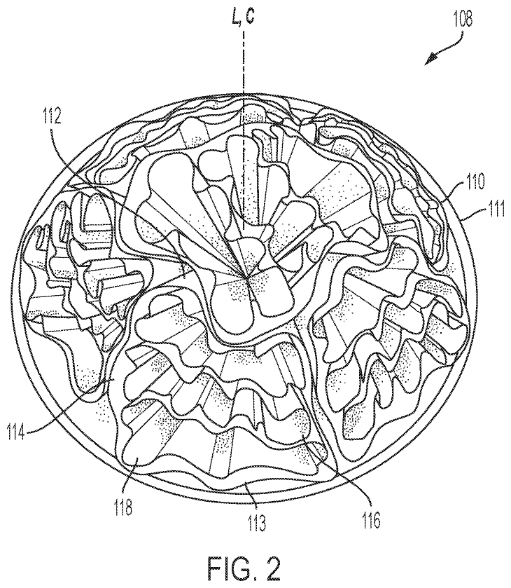

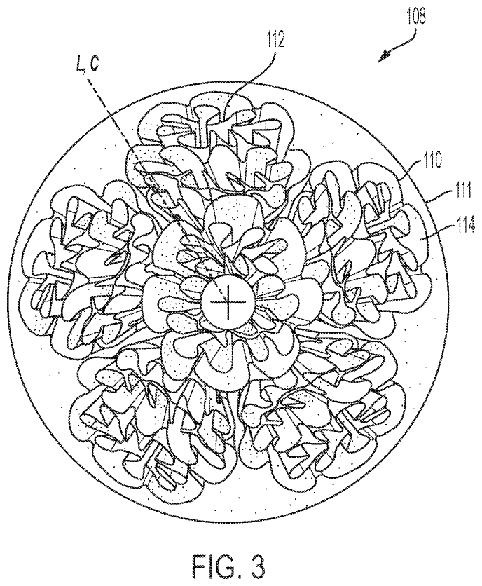

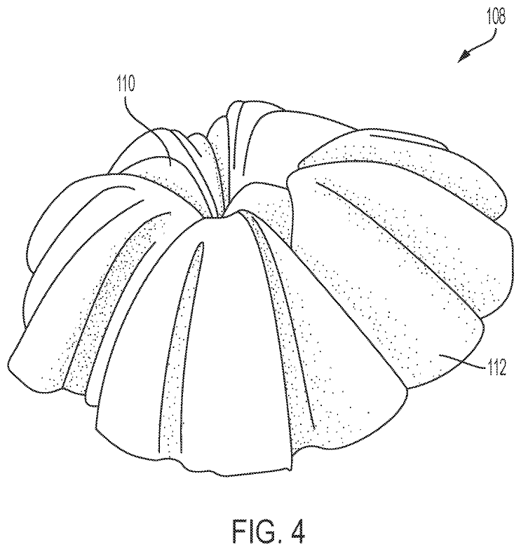

Turning now to the figures, FIGS. 2-5 illustrate a prosthesis 108 suitable for breast reconstruction and/or augmentation according to an embodiment of the present disclosure. As shown in at least FIGS. 2 and 3, the prosthesis may include a body 110 with a plurality of body components 112 (see also FIGS. 4 and 6-7) joined together to form an implant of a desired shape. The body components may be pre-formed to facilitate assembly of the prosthesis. In some embodiments, the prosthesis may include a base 111 that is attached to the prosthesis body, as described herein.

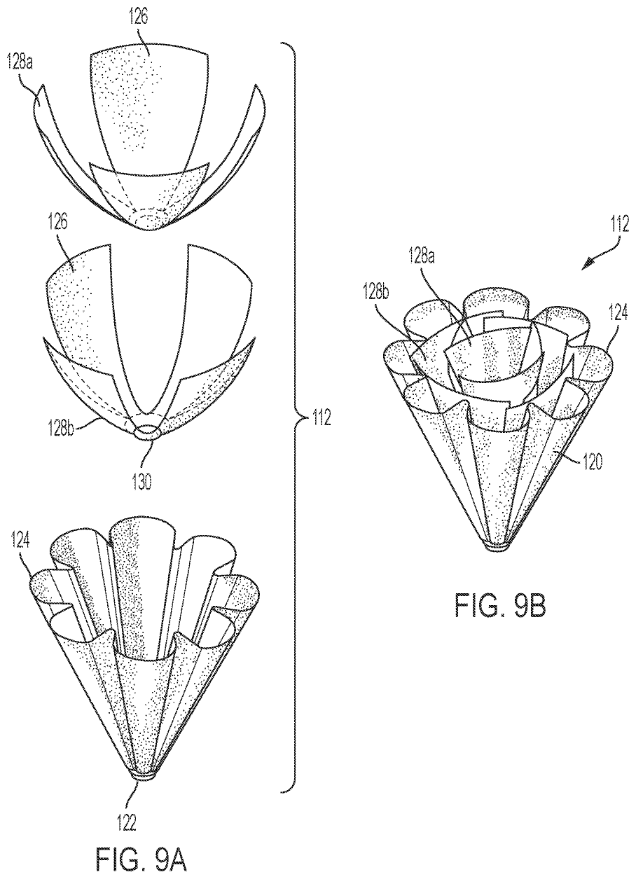

In some embodiments, as shown in FIGS. 2, 6, and 12, for example, each body component may have a first, open end 113 with a cavity 114 for receiving fat and/or tissue. In some embodiments, as described herein, one or more body components of the prosthesis may include a filler material 116 located within the cavity (see e.g., FIGS. 9A, 9B, and 15A-18B). In some embodiments, the filler material may add bulk to the prosthesis and/or create multiple voids 118 (see FIG. 2) within the respective cavity for receiving fat and/or tissue.

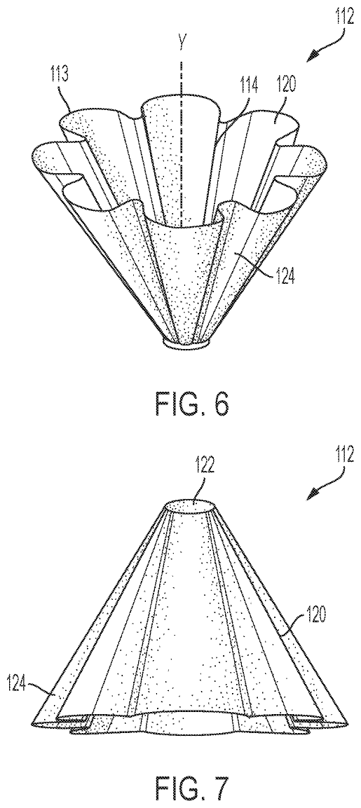

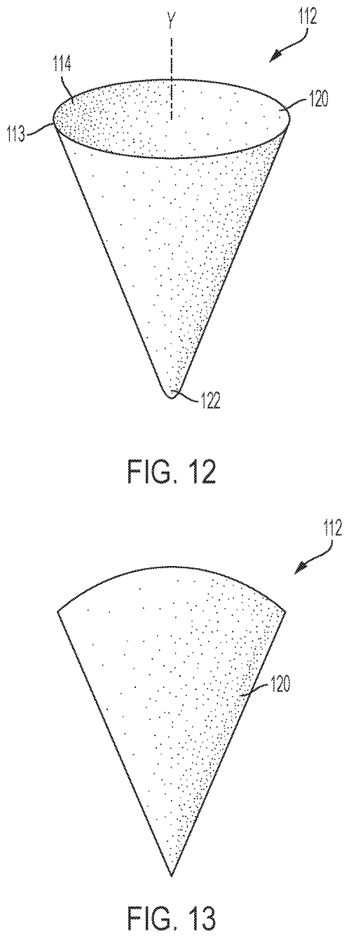

As shown in FIGS. 6 and 7, the body component may include an outer layer 120 which defines the cavity 114 for receiving fat and/or tissue. In some embodiments, as shown in FIGS. 6, 7, 12, and 14, the outer layer may have a generally conical shape. In some embodiments, as shown in FIG. 12, the outer layer may be symmetric about a longitudinal axis Y of the body component. In such embodiments, the filler material may have any suitable arrangement (e.g., pleated, not pleated, conical, etc.) such that the assembled body component may be symmetric or asymmetric about the longitudinal axis Y of the body component.

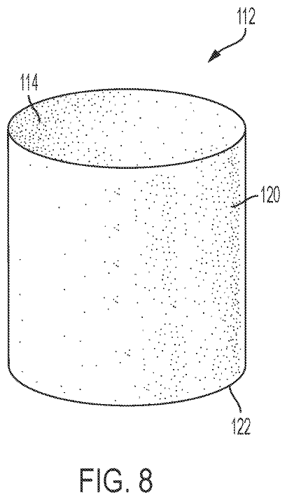

Although shown as having a generally conical shape, it will be appreciated that the outer layer may have other suitable shapes in other embodiments as should be apparent to one of skill in the art. For example, in some embodiments, the outer layer may be substantially cylindrical (see, for example, FIG. 8), rectangular, square, other polygonal, or other suitable shape.

As shown FIGS. 2, 6, 8, and 12, in some embodiments, the outer layer includes a second end 122 that is opposite the first, open end. In some embodiments, the second end 122 may be a closed end. In such embodiments, the filler material and/or the fat and/or tissue may be inserted into the body component via the open end. In some embodiments, the second, closed end may include a tip of the body component. In some embodiment, the body components may be joined to each other at their respective tips.

In some embodiments, the second end may be at least partially open. In some embodiments, the second end may be completely open. In some embodiments, the second end may be connected to another portion of the prosthesis (such that the second end is open in an unassembled position and closed when in an assembled position).

In some embodiments, a diameter of the open end of the body component may be greater than the diameter of the closed end. In some embodiments, the size of the body component may taper downwardly from the open end to the closed end. As will be appreciated, the diameter of the body component may decrease in other suitable ways. For example, the diameter may decrease in a stepped fashion in some embodiments. In other embodiments, such as when the body component is substantially cylindrical (see FIG. 8), the diameter of the open end and the closed end may be the same.

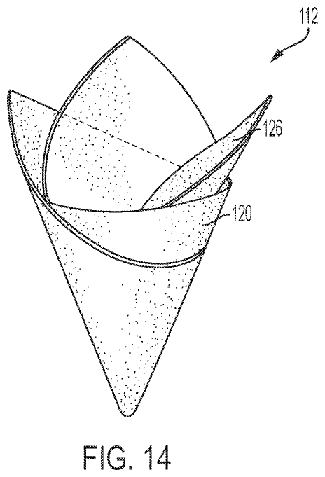

In some embodiments, as shown in FIGS. 12 and 14, the closed end of the prosthesis may include a generally conical tip. In other embodiments, as shown in FIGS. 6 and 7, the closed end may have a rounded or generally planar shape. In some embodiments, a planar closed end of the outer layer may extend substantially perpendicular to the longitudinal axis Y of the body component (see FIGS. 1, 2, and 6). As will be appreciated, the closed end may have other suitable shapes and arrangements in other embodiments as should be apparent to one of skill in the art.

In some embodiments, as shown in FIGS. 6 and 7, the outer layer of the body component may include pleats 124 to facilitate compressibility of the body component and/or to define voids on the outer surface of the body component. In some embodiments, the pleats may be arranged symmetric about the longitudinal axis Y of the body component. In other embodiments, the pleats may be arranged asymmetric about the longitudinal axis. As will be appreciated, the pleats may have any suitable shape and size. For example, the pleats may be U-shaped, V-shaped, or any other suitable shape as should be apparent to one of skill in the art.

As illustrated in FIGS. 9A-11, and 16A-18B, the body component may include filler material having different arrangements in various embodiments. As will be appreciated by one of skill in the art, the filler layers may have the same configuration or different configurations relative to each other. For example, and without limitation, separate filler layers may include different shapes, different sizes, different thickness, and/or any desired combinations thereof.

In some embodiments, as shown in FIGS. 9A and 9B, the filler material may include a plurality of petals 126 of material. In some embodiments, as illustrated, the filler material may include one or more petaled filler layers 128a, 128b, with each filler layer having a plurality of petals.

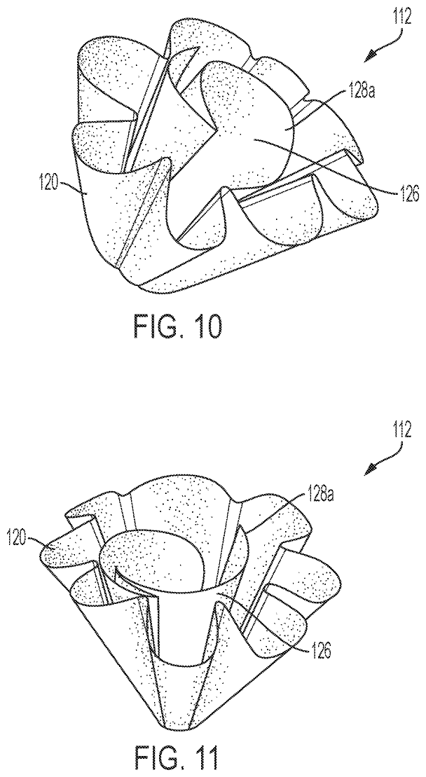

In some embodiments, as shown in FIGS. 10 and 11, the prosthesis may include one petaled filler layer 128a (e.g., one filler layer with petals) within the cavity of the outer layer. In other embodiments, as shown in FIGS. 9A and 9B, the prosthesis may include first and second petaled filler layers 128a, 128b within the cavity of the outer layer. As will be appreciated, the body component may include more than two petaled filler layers in other embodiments to achieve the desired shape and/or characteristics of the body component (e.g., a desired number of voids and/or a desired compressibility of the prosthesis).

In some embodiments, as shown in FIG. 9A, the petaled filler layer may include four petals 126. Alternatively, the petaled filler layer also may include more or fewer petals in other embodiments, as desired. For example, in some embodiments, as shown in FIGS. 9 and 10, the petaled filler layer may include two petals. The petaled filler layer also may include three petals, or more than four petals in other embodiments.

In some embodiments, the shape and size of the petals on the petaled filler layer may be the same. In other embodiments, the shape and size of each petal may vary from petal to petal. For example, in some embodiments, the petaled filler layer may include two relatively short petals and two relatively long petals. In some embodiments, the arrangement of the petals on each petaled filler layer may be the same. For example, as shown in FIG. 9A, each of the first and second petaled filler layers 128a, 128b may include four petals having the same shape and size. In other embodiments, the arrangement of the petals (e.g., shape, size, and number) on the petaled filler layers may vary from layer to layer. In some embodiments, as shown in FIG. 9B in the assembled configuration, the petals may be offset from one another. For example, in one embodiment, each filler layer may include four petals with the petals of one layer offset from the petals of another layer by an angle of approximately 90° about the longitudinal axis Y. In another embodiment, the petals also may be aligned in some embodiments. As will be appreciated, the petaled filler layer(s) may have different sized petals, different shaped petals, different spacing of petals, different materials, different thickness, different and/or any desired combination thereof in other embodiments.

In some embodiments, as shown in FIG. 9A, the petaled filler layers may be positioned in a stacked arrangement about the longitudinal axis of the body component in the assembled configuration. In other embodiments, the petaled filler layers may be positioned next to one another when in the cavity.

In some embodiments, the petaled filler layer may include a planar sheet of material with petals. In such embodiments, the petaled filler layer may have a 2D configuration prior to being assembled and a 3D configuration when assembled (see, for example FIG. 9A). In some embodiments, the petaled filler layer may be transformed from the 2D configuration to the 3D configuration by folding the planar sheet about a central portion, which, in the assembled configuration, may form a first end 130 (e.g., a closed end) of the petaled filler layer. In some embodiments, the petaled filler layer may be attached to the outer layer and/or to another petaled filler layer at the first end 130.

In some embodiments, the outer layer also may include one or more petals 126 (see FIG. 14). In such embodiments, the petals may be formed by folding one or more portions of the outer layer when forming the generally conical shape (or other suitable shape) of the body component. In some embodiments, the petals may extend outwardly and away from the open end of the cavity. As will be appreciated, the petals may have the same shape, size, thickness, and materials in some embodiments, although the shape, size, thickness, and materials may vary from petal to petal.

In some embodiments, the filler material also may include smaller body subcomponents inserted in the cavity formed by the outer layer. In some embodiments, the smaller body subcomponents may include a plug. For example, smaller conical layers, with or without pleats, may be inserted within the outer layer to increase bulk, increase void spaces and/or provide additional surface area for supporting fat and/or tissue. In some embodiments, the inner smaller body subcomponent may be between about 25% and 75% the size of the outer layer of the body component. The body component also may include other suitable filler materials in other embodiments as should be apparent to one of skill in the art.

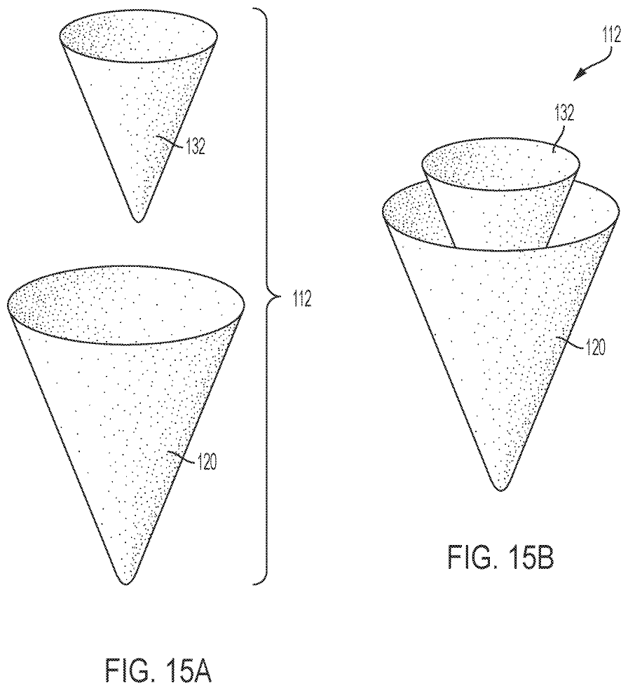

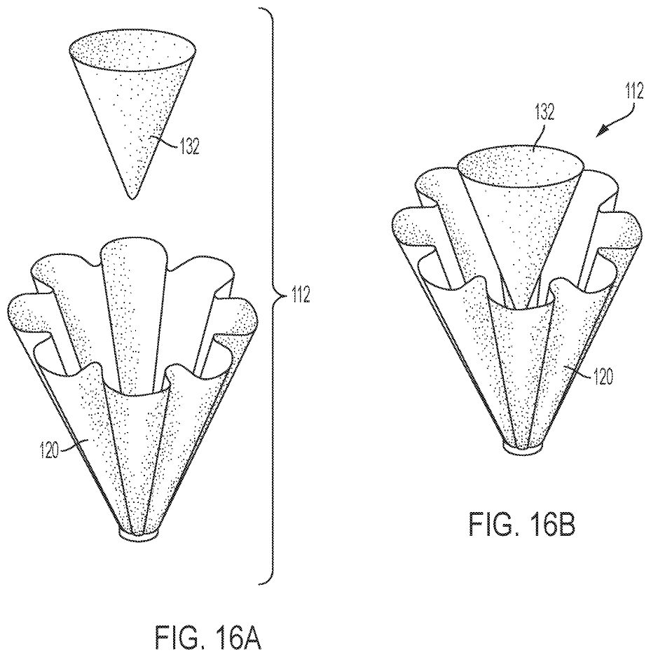

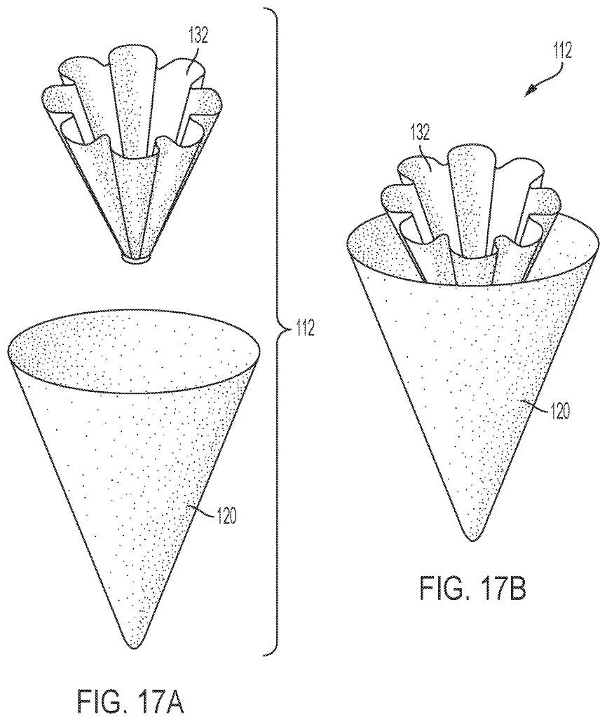

FIGS. 15A and 15B illustrate an embodiment in which the body subcomponent includes a smaller, conical inner filler layer 132 is inserted into a conical outer layer. As shown in these views, in some embodiments, the inner and outer layer may have the same configuration (e.g., shape). For example, the inner and outer layers may be substantially conical in shape with no pleats. In other embodiments, as illustrated in FIGS. 16A-17B, the inner and outer layers may have different configurations. For example, as shown in FIGS. 16A and 16B, the outer layer may be pleated while the inner layer is not pleated. As illustrated in FIGS. 17A and 17B, the inner filler layer 132 may be pleated while the outer layer 120 is not pleated. Although the inner and outer layers are both shown as being conical, it is to be appreciated that the inner and/or outer layer may employ other suitable shapes, and/or combinations of different shapes including, but not limited to, a conical shape for one layer and a non-conical shape for another layer.

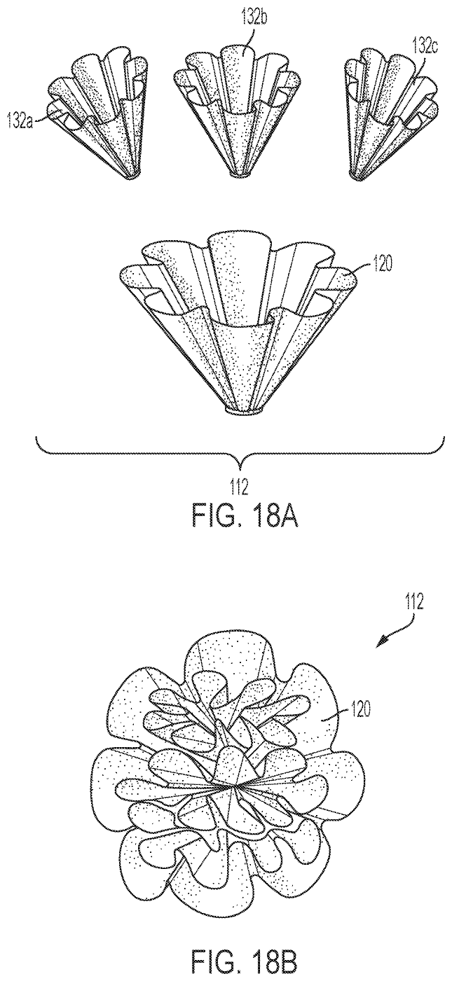

In some embodiments, the body subcomponent may include only a single inner filler layer (see, e.g., FIGS. 16A-17B). In other embodiments (see, for example, FIGS. 18A and 18B), the body component may include two or more inner filler layers. For example, in some embodiments, as shown in these figures, the body component may include three pleated filler layers or subcomponents within a cavity of a pleated outer layer. As will be appreciated in view of the above, in embodiments in which the body component includes more than one body subcomponent or filler layer, the filler layers may have the same arrangement. For example, as shown in FIG. 18A, each of the filler layers 132a-132c may have a conical shape and pleated. The filler layers also may have different arrangements in other embodiments. In some embodiments, the filler layer may include one or more smaller body components and a pleated filler layer.

In some embodiments, as shown in FIGS. 18A and 18B, the inner filler layers may be placed next to one another (e.g., adjacent to one another). In other embodiments, the inner filler layers may be stacked on each other along the longitudinal axis of the body component (e.g., similar to the nested inner petaled filler layers shown in FIGS. 9A and 9B).

In some embodiments, the body component may include a smaller inner body component and a petaled inner filler layer. For example, in some embodiments, a conical inner filler layer may be nested with one or more petaled filler layers.

In some embodiments, the body components and/or body subcomponents may include a plug. In one embodiment, the prosthesis may include body components and/or body subcomponents formed from PERFIX plugs and/or PHASIX plugs available from Davol, Inc.

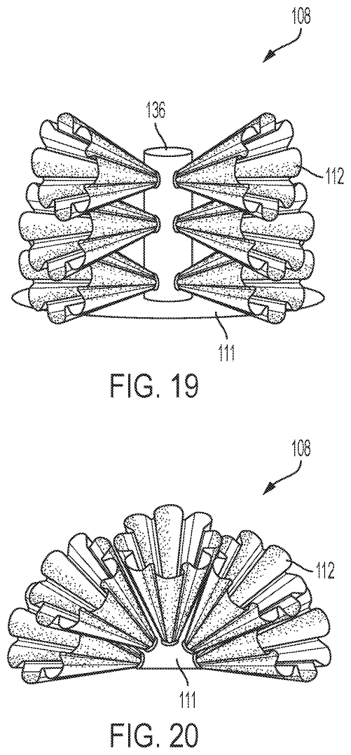

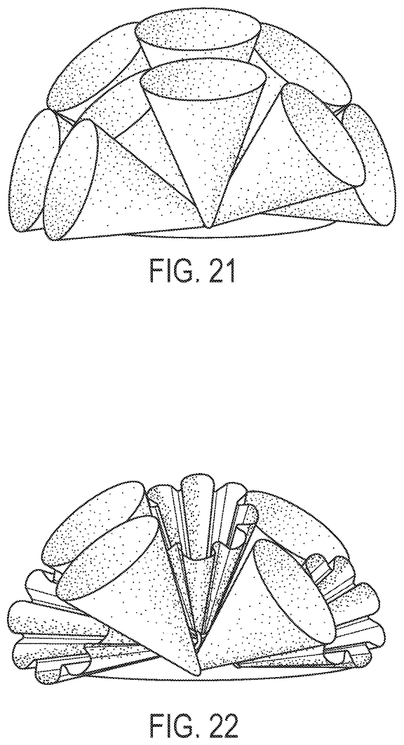

As described herein, when assembled, the body components may form a prosthesis having a generally hemispherical configuration. FIGS. 19-22 illustrate various embodiments of arrangements of body components attached together to form the prosthesis body. In some embodiments, as shown in FIGS. 20 and 21, for example, the body components may have the same configuration and be attached together to form the prosthesis body. In other embodiments, as shown in FIG. 22, the prosthesis body may be formed of body components having different configurations.

As shown in FIGS. 2 and 3, in some embodiments, the body components may be radially and/or circumferentially arranged about the longitudinal axis L of the prosthesis. For purposes herein, the longitudinal axis of the prosthesis also may define the longitudinal axis of the prosthesis body. In such embodiments, the second, closed ends (e.g., tips) of the body components may be located adjacent each other about the longitudinal axis L. As illustrated in these figures, the body components may be oriented with the open ends facing in an outward radial direction so that the cavity of each body component is oriented to face outwardly to accept and/or receive fat and/or tissue therein.

In such embodiments, the body components may be attached to one another at the longitudinal axis. In such embodiments, a hollow core may be formed via the arrangement of the body components about the longitudinal axis. In other embodiments, as shown in FIG. 19, the prosthesis may include a hollow core structure about which the plurality of body components may be attached. In such embodiments, a longitudinal axis of the hollow core structure may be aligned with the longitudinal axis of the prosthesis.

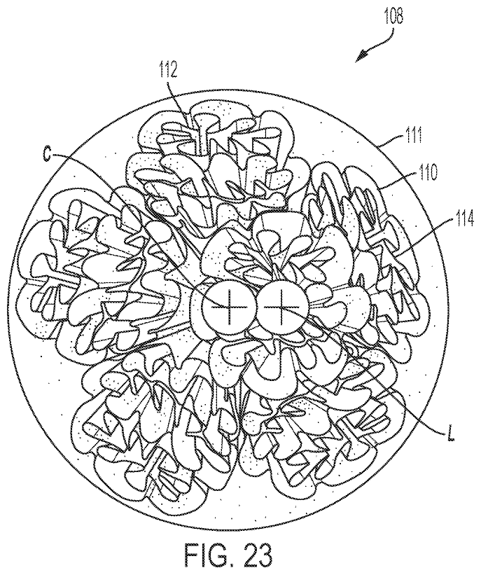

In some embodiments, as shown in FIGS. 2 and 3, the longitudinal axis may define a central axis C of the prosthesis. In other embodiments, as shown in FIG. 23 the longitudinal axis also may be spaced apart (e.g., offset) from the central axis C. In some embodiments, the longitudinal axis may be parallel to and spaced from the central axis.

In some embodiments, the prosthesis may include one or more tiers of body components. In such embodiments, each tier may include a plurality of body components arranged about the longitudinal axis of the prosthesis, with the closed ends of the body components adjacent each other. In some embodiments, the tiers may be stacked on one another along the longitudinal axis L of the prosthesis, with a first tier being adjacent to the base, a second tier stacked on top of the first tier, a third tier stacked on top of the second tier, and so on.

In some embodiments, each tier may include the same number of body components, although the tiers may include different numbers of body components. For example, as shown in FIGS. 2 and 3, the body prosthesis may include a first tier, adjacent the base, with five body components and a second tier, stacked on top of the first tier, with a single body component. In some embodiments, the body components of each tier may include the same configuration (e.g., shape and size), although the configuration of the body components may vary from tier to tier.

In embodiments having multiple tiers of body components, each tier may have body components arranged about the longitudinal axis of the prosthesis. In some embodiments, a top layer of the prosthesis body may have only a single body component, which may be substantially aligned with the longitudinal axis.

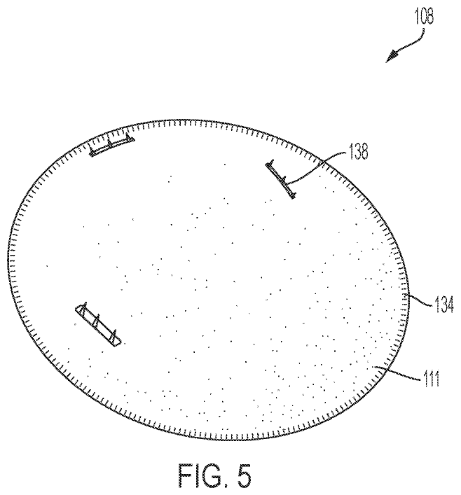

In some embodiments, the body components may be attached to each other, such as via the second end (e.g., tip) of the body component to form the prosthesis body. Other portions of adjacent body portions also may be attached to one another in other embodiments. In some embodiments, the body components may be attached to each other via stiches and/or ultrasonic welding. As will be appreciated, the body components may be attached to each other via other suitable ways. In some embodiments, the prosthesis body may be attached to the base, such as via stitches (see stiches 134 in FIG. 5). The prosthesis body also may be attachable to the base via other suitable methods, such as via ultrasonic welding.

In some embodiments, only a bottom tier of body components may be attached to the base. In some embodiments, all of the body components may be attachable to the base. For example, in some embodiments, the second ends of each body component may be attached to the base in some embodiments. As will be appreciated, other portions of the body components also may be attached to the base. In some embodiments, the body components may be attachable to the base via stitches, via ultrasonic welding, and/or via any other suitable methods. In some embodiments, the body components may be attached only to the base and not to each other.

In still other embodiments, the body components may be attached to other suitable portions of the prosthesis. For example, as shown in FIG. 19, the body components may be attached to and may be arranged radially and/or circumferentially about a hollow core structure 136. In some embodiments, the body components may be attached to the hollow core but not to one another.

In some embodiments, the hollow core may include a cylindrical structure, although the core may have other suitable structures. For example, in some embodiments, the hollow core may be square, rectangular, conical, or other suitable shapes.

In some embodiments, the hollow core may be attached to the base. In some embodiments, the base may include an annular shape such that the core is open at a bottom end. In such embodiments, the base may have an opening therethrough which is aligned with a first end of the hollow core to permit access to the hollow core through the base. In some embodiments, the top of the hollow core also may be open. In such embodiments, fat and/or tissue may travel into and around the hollow core.

In some embodiments, as show in FIGS. 2 and 5, for example, the base may be configured to provide a support for attaching the prosthesis in a desired position relative to the CML and/or adjacent tissue or muscle. The base may be configured to have any suitable shape and/or size conducive to positioning and/or fixating the prosthesis to adjacent tissue. In some embodiments, the base may include a sheet. The base may include one or more layers attached to one another in some embodiments.

In some embodiments, the bottom surface of the base may be planar. The bottom surface also may have other suitable configurations. For example, the bottom surface may have a generally rounded shape such as a convex shape.

Although the prosthesis body is shown and described as being attached to a base, in some embodiments, the prosthesis may include only a prosthesis body. In such embodiments, the body may be attached directly to the surrounding tissue and/or muscle (e.g., via stitches). In such embodiments, a bottom surface of the body may be configured for attachment to the surrounding fascia.

In some embodiments, as shown in FIG. 5, the base may include one or more grips 138 arranged to penetrate and grip adjacent tissue and/or muscle to facilitate positioning and/or fixation of the prosthesis to the adjacent tissue and/or muscle. In some embodiments, the grip(s) may extend outwardly from the bottom of the prosthesis.

In some embodiments, as shown in FIG. 5, the prosthesis may include three grips 138 although the prosthesis may include more or fewer grips in other embodiments. For example, the prosthesis may include, one, two, three, four, or more grips in other embodiments. In some embodiments, the grips may be positioned about a periphery of the bottom surface of the base. For example, the grips may be equally spaced about the periphery of the prosthesis. The grips may be positioned in other suitable locations in other embodiments. For example, the prosthesis may include a single, larger, grip positioned in center of the bottom of the prosthesis.

In some embodiments, each grip may include one or more teeth for positioning and/or fixing the prosthesis to the adjacent tissue and/or muscle. In some embodiments, the grip may include one or more staples, tacks, or other suitable grippers and/or fasteners. In some embodiments, as shown in FIG. 5, the grips may extend outwardly from the bottom of the prosthesis.

As will be appreciated in view of the present disclosure, the size of the prosthesis may be defined by the size, shape, and/or number of body components joined together. However, the desired size of a prosthesis may differ from patient to patient. Accordingly, for some applications, it may be desirable to adjust the size and/or bulk of the prosthesis by removing and/or adding material. For example, and without limitation, the size and/or bulk of the prosthesis may be reduced by removing one or more body components and/or removing a portion or all of the filler material from one or more of the body components.

In some embodiments, the prosthesis may be pre-formed, with the body components being assembled prior to a reconstruction procedure. For example, prior to reconstruction, the body components may be assembled to form a prosthesis body by attaching one or more body components to each other or to another suitable portion of the prosthesis, as described herein. In some applications, the pre-formed prosthesis may be trimmed to create a desired shape for implantation. For example, and without limitation, one or more body components (e.g., the outer layer and/or the filler material) may be trimmed to create a customized implant shape. In such an example, the body components may each be trimmed any desired amount depending upon the desired size and shape of the prosthesis for a particular application.

In other embodiments, the surgeon may add additional body components to the prosthesis to create the customized prosthesis shape. In such embodiments, the same body components may be added to the prosthesis body, although different body components can be added to the prosthesis body. In some embodiments, one or more body components may be trimmed before attachment to the prosthesis body.

In some embodiments, the prosthesis may not be pre-formed but instead may be assembled by the surgeon in conjunction with a reconstruction procedure (e.g., prior to or during a procedure) to create a customized implant shape. In such embodiments, the prosthesis may include a kit having multiple body components and/or other prosthesis components (e.g., a base and/or hollow core) that may be assembled by the surgeon. For example, the surgeon may select and attach one or more body components together or to other suitable components to achieve the customized implant shape. Each body component may have the same configuration or may have different configurations. In some embodiments, the surgeon also may trim one or more of the body components during assembly, as desired.

In some embodiments, the body components also may be pre-formed. In some embodiments, the body component may include a 2D configuration that is transformed into a 3D configuration before attaching to another body component, a base, or another suitable portion of the prosthesis body. In some embodiments, the 2D configuration may include a sheet (see FIG. 13), which may be opened to form the 3D configuration (see FIG. 12).

Various structural arrangements and/or manufacturing techniques may be employed to fabricate a relatively complex implant.

It may be desirable to provide a durable, light weight implantable prosthesis for breast reconstruction or augmentation without concern of long-term foreign material.

According to one aspect, the prosthesis may be fabricated from an absorbable material. In one embodiment, the prosthesis may be fabricated from a relatively slow absorbing material, such as P4HB (Poly-4-hydroxybutyrate), to provide long term support for the breast as fat and/or tissue eventually fill and replace the prosthesis to promote a more natural appearance and feel for the breast. The material may be sufficiently porous to promote passage of fat and/or ingrowth of tissue within the prosthesis, although a porous material is not required for each embodiment. The prosthesis may include knitted, woven and/or non-woven material.

In one embodiment, the prosthesis may be fabricated with PHASIX mesh, which is manufactured from P4HB, available from Davol, Inc. of Warwick, RI. Other suitable materials may include, but are not limited to, GalaFLEX available from Galatea, TIGR Matrix available from Novus Scientific, SERI Surgical Scaffold available from Allergen, BIO-A available from Gore, and ULTRAPRO available from Ethicon. If desired, a non-woven material, such as Phasix, may be employed as an alternative or together with a mesh to provide a relatively softer profile for the prosthesis. For some applications, it may be desirable to fabricate the prosthesis, or one or more portions of the prosthesis, from a non-absorbable material including, but not limited to, polypropylene and polytetrafluoroethylene (PTFE).

For some applications, it may be desirable to coat the prosthesis with material to provide one or more properties. For example, and without limitation, it may be desirable to minimize bleeding, minimize seroma formation and/or facilitate tissue ingrowth. In one embodiment, the prosthesis may be coated with Arista AH available from Davol, Inc.

For some applications, it may be desirable for the prosthesis to have a relatively smooth outer surface. According to one aspect, the 3D structure of the prosthesis may be covered with an outer layer of material including, but not limited to, P4HB or collagen. In such embodiments, the prosthesis may include an outer layer that overlies the prosthesis body and that corresponds with the shape of the prosthesis body. As will be appreciate, in other embodiments, one or more body components may be arranged to have a relatively smooth outer surface and may have a smooth outer layer. In such embodiments, the outer layer may be applied to the body component before attaching the body component to the prosthesis body.

It may be desirable for particular applications for the prosthesis to be constructed to provide a desired amount of resistance to permanent deformation after compression. In one embodiment, the prosthesis may be constructed to have a reduction in height or no more than 10% (i.e., ≤10%) of its original height H after being subjected to vertical compression of 40% of its height H. The reduction in height may be determined at one or more times following compression. In one embodiment, the reduction in height may be determined at time t=0 and at time t=12 weeks. However, it is to be appreciated that the prosthesis may be configured to provide any suitable amount of resistance to permanent deformation as should be apparent to one of skill in the art.

It may be desirable to provide an implantable prosthesis which can support the resected space along with transferred adipose tissue during healing and integration of the implant.

According to one aspect, the prosthesis may have a compressive strength to oppose biomechanical forces within the breast. In one embodiment, the implant may have a compressive strength of at least 3.1 lbf (i.e., ≥3.1 lbf) at 25% vertical compression at time t=0 and at least 2.4 lbf (i.e., ≥2.4 lbf) at 25% vertical compression at time t=12 weeks. However, it is to be appreciated that the prosthesis may be configured to have any suitable amount of compressive strength as should appreciated by one of skill in the art.

According to one aspect, the prosthesis may employ connections having a connection strength which is sufficient to maintain the mechanical integrity of the device. In one embodiment, the implant may employ connections having a connection strength of at least 1.0 lbf (i.e., ≥1.0 lbf) at time t=0. However, it is to be appreciated that the prosthesis may be configured to have any suitable amount of connection strength as should appreciated by one of skill in the art.

The overall desired structure of the prosthesis may employ various constructs for its fabrication in an efficient manner.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Aspects of the disclosure may also be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

For purposes of this patent application and any patent issuing thereon, the indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

1. An implantable prosthesis comprising:
a tissue infiltratable body of biocompatible material, the body including a proximal end and a distal end spaced from the proximal end, the body being configured to augment and/or reconstruct an anatomical shape of a human breast, the body including:
a plurality of body components circumferentially arranged about a longitudinal axis of the body extending from the proximal end to the distal end, each of the plurality of body components including an outer layer defining a cavity, the outer layer having a conical shape.

2. The implantable prosthesis of claim 1, wherein the prosthesis includes a base attached to the proximal end of prosthesis body.

3. The implantable prosthesis of claim 2, wherein each of the plurality of body components is attached to the base and/or to one another.

4. The implantable prosthesis of claim 1, wherein the outer layer of one or more of the plurality of body components is pleated.

5. The implantable prosthesis of claim 4, wherein the outer layer of at least two of the plurality of body components is pleated.

6. The implantable prosthesis of claim 1, wherein one or more of the plurality of body components includes a filler material within the cavity.

7. The implantable prosthesis of claim 6, wherein the filler material includes at least one filler layer.

8. The implantable prosthesis of claim 7, wherein the at least one filler layer includes a plurality of pleats and/or has a conical shape.

9. The implantable prosthesis of claim 7, wherein the at least one filler layer has a shape which corresponds with a shape of the outer layer of the body component.

10. The implantable prosthesis of claim 7, wherein the at least one filler layer includes a plurality of petals.

11. The implantable prosthesis of claim 1, wherein the body includes one or more tiers of body components, the one or more tiers being stacked along the longitudinal axis of the prosthesis.

12. The implantable prosthesis of claim 1, wherein each of the plurality of body components includes an open end with one or more voids facing an outward radial direction away from the longitudinal axis, the one or more voids configured to allow fat and/or tissue to fill and pass through the body and fill-out the implantable prosthesis.

13. The implantable prosthesis of claim 12, wherein each of the plurality of body components includes a closed end opposite the open end.

14. The implantable prosthesis of claim 1, wherein the plurality of body components are compressible and/or collapsible.

15. The implantable prosthesis of claim 1, wherein the plurality of body components are pre-formed.

16. The implantable prosthesis of claim 1, wherein the body includes a hollow core, the plurality of body components being arranged about the hollow core.

17. The implantable prosthesis of claim 16, wherein the hollow core is attached to a base attached to a lower end of the body, the base having an opening therethrough which is aligned with a first end of the hollow core to permit access to the hollow core through the base.

18. The implantable prosthesis of claim 1, wherein the longitudinal axis defines a central axis of the prosthesis.

19. An implantable prosthesis comprising:
a tissue infiltratable body of biocompatible material, the body having a proximal end and a distal end spaced from the proximal end, the body being configured to augment and/or reconstruct an anatomical shape of a human breast, the body including:
a plurality of body components arranged about a longitudinal axis of the body extending from the proximal end to the distal end, wherein each of the body components includes an outer layer defining a cavity, the outer layer being pleated; and
a base attached to the body.

20. The implantable prosthesis of claim 19, wherein one or more of the plurality of body components includes a first filler layer in the cavity.

21. The implantable prosthesis of claim 20, wherein the first filler layer includes a plurality of petals.

22. The implantable prosthesis of claim 20, wherein the first filler layer is substantially conically shaped and/or is pleated.

23. The implantable prosthesis of claim 20, wherein the first filler layer and the outer layer of the one or more of the plurality of body components are the same shape.

24. The implantable prosthesis of claim 19, wherein the body includes one or more tiers of body components, the one or more tiers being stacked along the longitudinal axis.

25. The implantable prosthesis of claim 19, wherein each of the plurality of body components includes an open end with one or more voids facing in an outward radial direction away from the longitudinal axis, the one or more voids arranged to allow fat and/or tissue to fill and pass through the body and fill-out the implantable prosthesis.

26. The implantable prosthesis of claim 25, wherein each of the plurality of body component includes a closed end opposite the open end.

27. The implantable prosthesis of claim 19, wherein the plurality of body components are compressible and/or collapsible.

28. The implantable prosthesis of claim 19, wherein the plurality of body components are pre-formed.

29. The implantable prosthesis of claim 19, wherein the outer layer of one or more of the plurality of body components is conical in shape.

30. The implantable prosthesis of claim 19, wherein the longitudinal axis defines a central axis of the prosthesis.

31. A method of fabricating an implantable prosthesis, the method comprising acts of:
(a) providing a plurality of body components, each of the plurality of body components including an outer layer defining a cavity, wherein:
the outer layer has a conical shape; and/or
the outer layer is pleated;
(b) arranging the plurality of body components circumferentially about a longitudinal axis of the prosthesis and in an outward radial direction to form a body configured to augment and/or reconstruct an anatomical shape of a human breast; and
(c) securing the plurality of body components together.

32. The method of claim 31, wherein act (c) includes attaching one or more portions of each body segment to one or more portions of an adjacent body segment.

33. The method of claim 31, wherein act (a) includes pleating the outer layer of one or more of the plurality of body components.

34. The method of claim 31, further comprising an act (d) of attaching one or more of the plurality of body components to a base.

35. The method of claim 31, wherein step (b) includes:
arranging a first plurality of body components about the longitudinal axis to form a first tier; and
arranging a second plurality of body components about the longitudinal axis to form a second tier wherein the second tier is stacked on the first tier along the longitudinal axis.

36. The implantable prosthesis of claim 31, wherein the longitudinal axis defines a central axis of the prosthesis.

37.-58. (canceled)
